SEQUENCE LISTING (3 sequences)

SEQ ID NO: 1 - protein, 127 aa, Synthetic construct
APARSPSPSTQPWEHVNAIQEAPRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 2 - protein, 127 aa, Synthetic construct
APARSPSPSTQPWEHVNAIQEALRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 3 - protein, 127 aa, Synthetic construct [sequence omitted]

(19) United States
(12) Patent Application Publication
(10) Pub. No.: US 2026/0295009 A1
(43) Pub. Date: Oct. 1, 2026

(54) HUMORAL MODULATION IN VACCINATED AND VIRALLY INFECTED SUBJECTS WITH GRANULOCYTE-MACROPHAGE COLONY-STIMULATING FACTOR (GM-CSF)

(71) Applicant: PARTNER THERAPEUTICS, INC., Lexington, MA (US)

(72) Inventors: Ila JOSHI, Lexington, MA (US); Fiona GARNER, Lexington, MA (US)

(21) Appl. No.: 19/480,710

(22) PCT Filed: May 2, 2024
(86) PCT No.: PCT/US2024/027377

§ 371 Date: Oct. 31, 2025

Related U.S. Application Data
(60) Provisional application No. 63/463,762, filed on May 3, 2023.
(60) Provisional application No. 63/586,444, filed on Sep. 29, 2023.

(51) Int. Cl. A61K 38/19; A61K 31/403; A61K 31/427; A61K 31/573; A61P 31/14; A61P 37/04; C07K 16/104

(52) U.S. Cl. CPC A61K 38/193; A61K 31/403; A61K 31/427; A61K 31/573; A61P 31/14; A61P 37/04; C07K 16/104

(57) ABSTRACT

The present disclosure relates to the treating or preventing a viral infection in a subject who has received a viral vaccine with granulocyte-macrophage colony-stimulating factor.

### CROSS REFERENCE TO RELATED APPLICATIONS

This application claims priority to U.S. Provisional Patent Application Nos. 63/463,762, filed May 3, 2023, and 63/586,444, filed Sep. 29, 2023, the entire contents of each of which is hereby incorporated by reference for all purposes.

### FIELD

This disclosure relates to, in part, treatment and/or mitigation of viral infections, including the humoral modulation in vaccinated and infected patients.

### SEQUENCE LISTING

This application contains a Sequence Listing in XML format submitted electronically herewith via Patent Center. The contents of the XML copy, created on Apr. 28, 2024, is named “PNR-014PC_127114-5014.xml” and is 4,068 bytes in size. The Sequence Listing is incorporated herein by reference in its entirety.

### BACKGROUND

Many, if not all, viruses, including respiratory viruses such as influenza, respiratory syncytial virus, rhinovirus and coronavirus, suppress innate immune responses to gain a window of opportunity for efficient virus replication and establishment of an infection. The consequences for the host's immune response are that it is often incomplete, delayed or diminished, or displays overly strong induction (after the delay) that may cause tissue damage. Kikkert M et al. J Innate Immun. 2020 January; 12(1): 4-20. Seemingly mild cases of COVID-19 have been shown to rapidly worsen into severe cases that involve the lower lungs. It is thought that the rapid worsening of COVID-19 cases may be, in part, attributable to “the cytokine storm”, which is an overproduction of immune cells and their activating compounds—cytokines or chemokines—and is often associated with a surge of activated immune cells into the lungs and a systemic inflammatory insult involving many organs. In the lung, the resulting lung inflammation and edema (fluid buildup) can lead to respiratory distress and can be worsened by a secondary bacterial pneumonia, which increases the risk of mortality in patients. These patients can progress rapidly with acute respiratory distress syndrome (ARDS) and septic shock, followed by multiple organ failure. Identification and treatment of this hyperinflammation is important to reduce the rising mortality. Mehta et al. Lancet. 2020 Mar. 28; 395 (10229): 1033-1034. Most patients present with fever, dry cough, dyspnea, and bilateral ground-glass opacities on chest CT scans. Reports from COVID-19 patients has shown that high amounts of IL-1β, IFNγ, IP10, and MCP1 may be leading to activated T-helper-1 (Th1) cell responses. Critical COVID-19 patients also initiated increased secretion of T-helper-2 (Th2) cytokines (e.g., IL-4 and IL-10), probably as a counter measure to suppresses inflammation, further associating the cytokine storm with disease severity. Additionally, laboratory results from the patients have also identified an increase in IL-6 among other cytokines as a risk factor of cytokine storm in COVID-19-infected pneumonia patients. Cheung C Y et al. 2005. J Virol 79 (12): 7819-7826; Huang C, et al. Lancet 2020; 395:497-506; Chen N, et al. Lancet 2020; 395:507-13; Ruan et al. Intensive Care Med. 2020 Mar. 3. doi: 10.1007/s00134-020-05991-x.

The mechanism of host defense against viral infections is a symphony of many nonspecific and specific immunologic components. The relative restrictions of viral multiplication manifested by several host species is independent of antigen-specific immunity and appears to be determined by genetically controlled factors. Such resistance to viral infections may be related to failure of virus attachment to cell-membrane receptors, inefficient release of viral nucleic acid from infected cells, and defective assembly of viral structural proteins. The body also has a range of structural and chemical barriers that can protect from infectious agents. Beyond structural chemical barriers that can determine susceptibility to infection, the immune system can be simplistically viewed as having two “lines of defense”: innate immunity and adaptive immunity. The innate immunity represents the first line of defense to an intruding pathogen and is antigen-independent (non-specific) defense mechanism that is used by the host immediately or within hours of encountering an antigen. The innate immune response has no immunologic memory and, therefore, it is unable to recognize or “memorize” the same pathogen should the body be exposed to it in the future. On the other hand, the adaptive immunity is antigen-dependent and antigen-specific and, therefore, involves a lag time between exposure to the antigen and maximal response. The hallmark of adaptive immunity is the capacity for memory which enables the host to mount a more rapid and efficient immune response upon subsequent exposure to the antigen. Importantly, the innate and adaptive immunity are not mutually exclusive mechanisms of host defense, but rather are complementary, with defects in either system resulting in host vulnerability or inappropriate responses. Numerous cells are involved in the innate immune response such as phagocytes (macrophages and neutrophils), dendritic cells, mast cells, basophils, eosinophils, natural killer (NK) cells and innate lymphoid cells. The cells of the adaptive immune system include antigen-specific T cells, which are activated to proliferate through the action of APCs, and B cells which differentiate into plasma cells to produce antibodies or immunoglobulins. See Ogra P L et al. Viral Immunology and Immunopathology. 1975. 57-77; Murphy K M et al. Janeway's Immunobiology. 7th ed. New York: Garland Science; 2007; Turvey S E and Broide D H. J Allergy Clin Immunol. 2010; 125 (Suppl 2):S24-32; Bonilla F A and Oettgen H C. J Allergy Clin Immunol. 2010; 125 (Suppl 2):S33-40; Marshall J S et al. Allergy Asthma Clin Immunol 2018, 14 (Suppl 2): 49.

The characteristics of a specific viral antibody response appear to be intimately related to the natural pathogenesis of the viral infection, the nature of available immunocompetent tissue, the route of infection, prior antigenic exposure, and many other parameters of host-virus interaction. A major component of humoral immune response to viral infections is the development of secretory antibody at mucosal portals of entry. The secretory antibody response when stimulated locally is quite independent of the systemic immunologic responses. Mucosal antibody response can be expected after infection or immunization with replicating or nonreplicating viral agents, regardless of the route of administration, provided adequate amounts of viral antigen became available to the mucosal immunocompetent tissues. Further, unlike T cells, B cells can recognize antigens directly, without the need for APCs, through unique antibodies expressed on their cell surface. The principal function of B cells is the production of antibodies against foreign antigens which requires their further differentiation. When activated by foreign antigens to which they have an appropriate antigen specific receptor, B cells undergo proliferation and differentiate into antibody-secreting plasma cells or memory B cells. Memory B cells are “long-lived” survivors of past infection and continue to express antigen-binding receptors. These cells can be called upon to respond quickly by producing antibodies and eliminating an antigen upon re-exposure. Plasma cells, on the other hand, are relatively short-lived cells that often undergo apoptosis when the inciting agent that induced the immune response is eliminated. Five major types of antibodies are produced by B cells: IgA, IgD, IgE, IgG and IgM. IgG antibodies can be further subdivided into structurally distinct subclasses with differing abilities to fix complement, act as opsonins, etc. The major classes of antibodies have substantially different biological functions and recognize and neutralize specific pathogens. Defects or malfunctions in either the innate or adaptive immune response can provoke illness or disease. Such disorders are generally caused by an overactive immune response (known as hypersensitivity reactions), an inappropriate reaction to self (known as autoimmunity) or ineffective immune responses (known as immunodeficiency). See Ogra P L et al. Viral Immunology and Immunopathology. 1975. 57-77; Murphy K M et al. Janeway's Immunobiology. 7th ed. New York: Garland Science; 2007; Turvey S E and Broide D H. J Allergy Clin Immunol. 2010; 125 (Suppl 2):S24-32; Bonilla F A and Oettgen H C. J Allergy Clin Immunol. 2010; 125 (Suppl 2):S33-40; Marshall J S et al. Allergy Asthma Clin Immunol 2018, 14 (Suppl 2): 49.

IgG4 is a subclass of IgG antibody with a unique molecular feature of (Fragment antigen-binding) Fab-arm exchange, allowing bispecific antigen binding in a mono-valent manner. IgG4 as a subclass detected recently and constitutes about 5% of total IgG, the smallest portion among all IgGs in serum. Although these different subtypes of IgG have more than 90% identical amino acids, they display different immunological effects such as immune complex formation and complement activation. Previous studies have revealed that different IgG subclasses are associated with different antigens. IgG1 and IgG2 subclasses are usually associated with the response to bacterial polysaccharides. IgG3 is a potent pro-inflammatory antibody to induce effector function, while IgG4 is usually associated with non-microbial allergens. IgG4 levels may vary significantly in healthy individuals, strongly hindering its clinical application as a diagnostic tool. Recent studies have shown that IgG4 levels can be significantly increased post-vaccination with COVID vaccines (e.g., mRNA vaccines). In particular, a boost in IgG4 levels were seen following the second and third booster vaccines and breakthrough COVID infection. Although this isotype class switching to a more non-inflammatory antibody response might decrease an antibody-dependent cellular phagocytosis and complement deposition-mediated immune response, it might serve as a counterbalance to an over-reaction of the immune response to SARS-Cov2 infection which could lead otherwise to significant morbidity (e.g., cytokine storm, ARDS) in these patients. It stands to reason that IgG4's non-inflammatory and tolerance-inducing functions could be considered to play a protective role in the context of some viral infections such as SARS-CoV2. See Aalberse R C et al. Clin Rev Allergy (1983) 1(2): 289-302; Van Milligen F J et al. Clin Exp Allergy (1995) 25(3): 247-51; Nirula A et al. Curr Opin Rheumatol (2011) 23 (1): 119-24; Vidarsson G et al. Front Immunol (2014) 5:520; Qin L et al., Front Immunol. 2022 Oct. 25; 13:1032909; Irrgang P et al. Sci Immunol. 2023.

Vaccinations have made an enormous and irreplicable impact on global health. Global coverage of vaccination against many important infectious diseases of childhood has been enhanced dramatically since the creation of WHO's Expanded Programme of Immunization in 1974 and of the Global Alliance for Vaccination and Immunization in 2000. Vaccines have been instrumental in the reduction in mortality and number of cases (e.g., flu, COVID), and in some cases the eradication of some of the world's deadliest diseases caused by viral and bacterial pathogens such as polio, diphtheria, pertussis, and tetanus, measles, mumps, rubella, hepatitis B, hepatitis A, Haemophilus influenzae type b, whooping cough (pertussis), pneumococcal disease, rotavirus and chickenpox). Vaccinations work by “training” the immune response to respond and mount a specific and robust response against an invading pathogen. Vaccinations against disease like coronavirus disease 2019 (COVID-19) have undoubtedly conferred widespread protection against infection worldwide and are strongly associated with prevention of serious illness, with a hospitalization rate 10.5 times higher in unvaccinated compared with fully vaccinated persons. See Plotkin S A. Nat Med. 2005 11(suppl 4):S5-S11; Greenwood B. Philos Trans R Soc Lond B Biol Sci. 2014. 369 (1645): 20130433; Accordino S et al. Eur J Intern Med. 2023. 111:124-126.

Colony Stimulating Factor (CSF) refers to a family of four glycoproteins that control and coordinate cell production by widely scattered deposits of marrow cells. These include Granulocyte-Macrophage CSF (GM-CSF), Granulocyte colony CSF (G-CSF), Macrophage colony CSF (M-CSF) and multipotential colony-stimulating factor (IL-3). These lymphokines can induce progenitor cells found in the bone marrow to differentiate into specific types of mature blood cells. The particular type of mature blood cell that results from a progenitor cell depends upon the type of CSF present. See Metcalf D. Cancer Immunol Res. 2013, 1 (6): 351-356.

GM-CSF is a hematologic growth factor that regulates the production, migration, proliferation, differentiation and function of hematopoietic cells. In response to inflammatory stimuli, GM-CSF is released by various cell types including T lymphocytes, macrophages, fibroblasts and endothelial cells. GM-CSF then activates and enhances the production and survival of neutrophils, eosinophils, and macrophages. Native GM-CSF is usually produced near the site of action where it can modulate proliferation, differentiation, and survival of hematopoietic progenitor cells. It is present in circulating blood in only picomolar concentrations (10⁻¹⁰ to 10⁻¹² M). See Alexander W S. Int Rev Immunol. 1998, 16:651-682; Gasson J C. Blood. 1991, 77:1131-1145; Shannon M F et al. Crit Rev Immunol. 1997, 17:301-323, Barreda D R et al. Dev Comp Immunol. 2004, 28:509-554 and Metcalf D. Immunol Cell Biology. 1987, 65:35-43.

Recombinant human granulocyte-macrophage colony-stimulating factor (rhGM-CSF) has been approved by the FDA for the treatment of neutropenia, blood dyscrasias and malignancies like leukemia in combination with chemotherapies. In the clinic, GM-CSF used for treatment of neutropenia and aplastic anemia following chemotherapy greatly reduces the risk of infection associated with bone marrow transplantation. Its utility in myeloid leukemia treatment and as a vaccine adjuvant is also well established. See Dorr R T. Clin Therapeutics. 1993. 15(1): 19-29; Armitage J O. Blood 1998, 92:4491-4508; Kovacic J C et al. J Mol Cell Cardiol. 2007, 42:19-33; Jacobs P P et al. Microbial Cell Factories 2010, 9:93.

Although there are five classes of heterologous protein production platforms, including bacteria, yeasts, plants, insect cells, and mammalian cells, more than 50% of currently marketed biopharmaceuticals are produced in mammalian cell lines. This is in part due to the inability of the remaining four classes to modify glycoproteins with human-like oligosaccharides. This is of importance since protein-bound glycans influence circulation half-life, tissue distribution, biological activity and immunogenicity. LEUKINE is a yeast-derived recombinant humanized granulocyte-macrophage colony stimulating factor (rhuGM-CSF, sargramostim) and the only FDA-approved GM-CSF.

The interaction between dendritic cells (DC) and naïve T cells is the first step in the evolution of an immune response, either tolerogenic or inflammatory. Therefore, the status of DC residing at mucosal sites, such as the airway, has a definitive impact on the character of the ensuing immune response. In the absence of pathogenic stimulation, DC serve to regulate immunological homeostasis in the lung; the generation of Th2-associated (allergic) inflammatory responses, which are directed at presumably innocuous antigens, represent a deviation from normal DC function. The dysregulation of DC phenotype leading to the development of a dysfunctional immune response might be programmed by genetic pedigree or might be induced by factors released in the airway. Given the pluripotent nature of GM-CSF, it could be used to condition DC to propagate Th2 responses. See Gajewska B U et al. Curr Drug Targets Inflamm Allergy. 2003 December; 2 (4): 279-92. Chatila T A et al. J Clin Invest. 2000; 106: R75-R81; Torgerson T R et al. Gastroenterology. 2007; 132:1705-17; Palomares O et al. Eur J Immunol. 2010; 40:1232-40; Jones S M et al. J Allergy Clin Immunol. 2014; 133:318-23; Palomares O et al. Genes Immun. 2014; 15:511-20; Rivas M N and Chatila T A J Allergy Clin Immunol. 2016 September; 138 (3): 639-652.

A therapeutic that might be complementary and that could synergistically act to enhance anti-viral therapies (e.g., viral vaccines), could be of immense clinical and economical value. Thus, there is further need for the development of such therapies that could prevent and/or mitigate infections to viruses.

### SUMMARY

Accordingly, in aspects, the present disclosure relates to a method for treating a vaccinated patient with a breakthrough viral infection, administering an effective amount of a composition comprising a granulocyte-macrophage colony-stimulating factor (GM-CSF) agent, wherein the effective amount is sufficient to induce a significant humoral response and a reduction in viral load, e.g., a clinically significant reduction of the viral load, and/or viral infection symptoms.

In aspects, the present disclosure relates to a method for treating or preventing a breakthrough viral infection, comprising selecting a patient who has received a vaccination to the infection and has one or more symptoms of the viral infection; and administering with an effective amount of a granulocyte-macrophage colony-stimulating factor (GM-CSF) agent, wherein the effective amount of GM-CSF is sufficient to modulate humoral immunity in the patient.

### BRIEF DESCRIPTION OF THE DRAWINGS

FIG. 1A depicts a randomized, open label clinical trial design.

FIG. 1B shows the type of COVID vaccine received by patients in the clinical trial design.

FIG. 1C shows the patient vaccination status in the biomarker study. Regarding the Analysis Population/Set of The “Vaccinated” Population/Set designation refers to patients who are at least two weeks post completion of COVID-19 vaccine regimen or participated in a COVID-19 vaccine clinical study. The Biomarker Analysis Set includes all randomized patients who consent to provide additional samples for biomarker assessment and who receive at least one dose of any study treatment. Analyses are performed according to the treatment received on Day 1. The Biomarker Analysis Set also includes all samples contributed by each patient in the Biomarker Analysis Population. Regarding the Biomarker Analysis Set, the number of subjects for each day is equal to the number of subjects in the analysis set on that day based on final disposition dates.

FIG. 2A depicts a graph that shows the anti-SARS-COV2 immunoglobulin (lg) reactivity from baseline in both sargramostim and placebo arms in all patients in the trial.

FIG. 2B depicts a graph that shows the anti-SARS-COV2 immunoglobulin (lg) reactivity from baseline in both sargramostim and placebo arms in the vaccinated patients in the trial.

FIG. 3A depicts the serum IgM titers against SARS-COV-2 antigens in vaccinated as compared to unvaccinated subjects.

FIG. 3B depicts the serum IgG titers against SARS-COV-2 antigens in vaccinated as compared to unvaccinated subjects.

FIG. 3C depicts the serum IgG4 titers against SARS-COV-2 antigens in vaccinated as compared to unvaccinated subjects.

FIG. 4A shows a table depicting the overall symptom score change from baseline in patients with and without treatment with Sargramostim.

FIG. 4B shows a graph of the symptom scores over 28 days in vaccinated-patients vs. unvaccinated-patients with and without treatment with Sargromostim. For orientation, the open red star line depicts the unvaccinated-sargramostim treated patients, the open diamond line depicts the vaccinated-sargramostim treated patients, the colored circle line depicts the unvaccinated-placebo treated patients, the open circle line depicts the vaccinated-placebo treated patients.

FIG. 5 shows a graph depicting IgG4 antibody profile in vaccinated vs. unvaccinated patients. For orientation, for each condition on the x-axis (i.e. “Ancestral trimer,” “Delta trimer,” “Gamma P.1 trimer,” “Beta trimer,” and “Alpha trimer”) there are four histograms, which are left to right: “Vaccinated-Placebo vs Unvaccinated-Placebo”, “Vaccinated-Sagramostim vs Unvaccinated-Sagramostim”, “Unvaccinated-Sagramostim vs Unvaccinated-Placebo,” “Vaccinated-Sagramostim vs Vaccinated-Placebo”.

### DETAILED DESCRIPTION

The present disclosure relates to, in part, the finding that granulocyte-macrophage colony-stimulating factor (GM-CSF) combined with a viral vaccine is an effective agent for treating and/or preventing a breakthrough viral infection, including, for example, decreasing the viral load and/or specifically modulating the immune response to the virus by inducing a significant humoral response.

Accordingly, in aspects, the disclosure provides methods for treating a patient immunized with a viral vaccine and has a breakthrough viral infection (e.g., a coronavirus infection).

### Coronavirus

The coronavirus is a member of the family Coronaviridae, including betacoronavirus and alphacoronavirus respiratory pathogens that have relatively recently become known to invade humans. The Coronaviridae family includes such betacoronavirus as Severe acute respiratory syndrome coronavirus 2 (SARS-COV-2), SARS-COV, Middle East Respiratory Syndrome-Corona Virus (MERS-COV), HCoV-HKU1, and HCoV-OC43. Alphacoronavirus includes, e.g., HCoV-NL63 and HCoV-229E.

Coronaviruses invade cells through “spike” surface glycoprotein that is responsible for viral recognition of Angiotensin Converting Enzyme 2 (ACE2), a transmembrane receptor on mammalian hosts that facilitate viral entrance into host cells. Zhou et al., A pneumonia outbreak associated with a new coronavirus of probable bat origin. Nature 2020. A new coronavirus infection 2019 (COVID-19), caused by

SARS-COV-2 causes severe respiratory distress, and this RNA virus strain has been the cause of the recent outbreak that has been declared a major threat to public health and worldwide emergency. Phylogenetic analysis of the complete genome of SARS-COV-2 revealed that the virus was most closely related (89.1% nucleotide similarity) to a group of SARS-like coronaviruses (genus Betacoronavirus, subgenus Sarbecovirus). Wu et al., A new coronavirus associated with human respiratory disease in China. Nature, Feb. 3, 2020.

The complete genome of the SARS-COV-2 coronavirus (29903 nucleotides, single-stranded RNA) is described in the NCBI database as GenBank Reference Sequence: MN908947. The coronavirus protein can be selected from the group consisting of: coronavirus spike protein (GenBank Reference Sequence: QHD43416), coronavirus membrane glycoprotein M (GenBank Reference Sequence: QHD43419), coronavirus envelope protein E (GenBank Reference Sequence: QHD43418), and coronavirus nucleocapsid phosphoprotein E (GenBank Reference Sequence: QHD43423).

The SARS-COV-2 is an enveloped, single stranded, RNA virus that encodes a “spike” protein, also known as the S protein, which is a surface glycoprotein that mediates binding to a cell surface receptor; an integral membrane protein; an envelope protein, and a nucleocapsid protein. The S protein, comprising S1 subunit and S2 subunit, is a trimeric class I fusion protein that exists in a prefusion conformation that undergoes a structural rearrangement to fuse the viral membrane with the host-cell membrane. See, e.g., Li, F. Structure, Function, and Evolution of Coronavirus Spike Proteins. Annu. Rev. Virol. 3:237-261 (2016), which is incorporated herein by reference in its entirety. The structure of the SARS-COV-2 spike protein in the prefusion conformation has been discovered. See Daniel et al., Cryo-EM structure of the SARS-COV-2 spike in the prefusion conformation. Science, 19 Feb. 2020, which is incorporated herein by reference in its entirety.

In embodiments, the method treats a breakthrough with SARS-COV-2 variants of concern (VOC), wherein the variant of SARS-COV-2 are selected from alpha (B.1.1.7), beta (B.1.351), gamma (P.1), delta (B.1.617.2), epsilon (B.1.427 or B.1.429), eta (B.1.525), iota (B.1.526), kappa (B.1.617.1), lambda (C.37), mu (B.1.621 or B.1.621.1), omicron (BA.1, B.1.1.529, BA.2, BA.2.75, BA.3, BA.4/BA.5, BN.1BQ.1, XBB, XBB.1.5, XAK, XAY, XBC), and zeta (P.2).

### Influenza Virus

The influenza virus is a member of the family Orthomyxoviridae, which include enveloped viruses with segmented negative-sense single-strand RNA segments. There are several genera of this family including types A, B, C, D and Thogotovirus, of which, however, only genera A and B are clinically relevant for humans. See Untergruppe A B. Transfus Med Hemother. 2009. 36 (1): 32-39.

The eight genome segments of influenza A and B viruses are loosely encapsidated by the nucleoprotein. The polymerase complexes consisting of the three polymerase proteins PB1, PB2, and PA are located at the ends of the nucleocapsids. These helical capsids are encircled by the M1 matrix protein and by a host-derived lipid bilayer envelope in which the virus surface glycoproteins haemagglutinin (HA) and neuraminidase (NA) as well as the M2 matrix protein are embedded. In embodiments, the influenza virus is selected from Type A, Type B, Type C, and Type D influenza.

In embodiments, the breakthrough viral infection is pandemic 2009 influenza A (H1N1) or avian influenza A (H5N1).

### Viral Infections and Breakthrough Virus Infections

In aspects, the disclosure provides methods for treating a breakthrough viral infection in a patient immunized to the virus.

In embodiments, the breakthrough viral infection is a respiratory viral infection.

In embodiments, the breakthrough viral infection is an infection with a coronavirus. In some embodiments, the breakthrough viral infection is an infection with an influenza virus.

In embodiments, the patient has experienced one or more prior infections by or exposures to the virus. In some embodiments, the patient has experienced one prior infection by or exposure to the virus. In some embodiments, the patient has experienced two or more prior infections by or exposures to the virus. In other embodiments, the patient has experienced three or more prior infections by or exposures to the virus. In yet other embodiments, the patient has experienced one or more prior infections by or exposures to the virus of at least about two weeks before the breakthrough infection.

In embodiments, the patient has mild or moderate COVID-19.

In embodiments, the patient is afflicted with one or more of fever, cough, shortness of breath, diarrhea, upper respiratory symptoms, lower respiratory symptoms, pneumonia, and acute respiratory syndrome. In some embodiments, the patient is afflicted with fever, tiredness, dry cough, aches and pains, shortness of breath and other breathing difficulties, diarrhea, upper respiratory symptoms (e.g., sneezing, runny nose, nasal congestion, cough, sore throat), pneumonia, pneumonia respiratory failure, hepatic and renal insufficiency, acute respiratory distress syndrome (ARDS), and a cytokine imbalance by one or more symptoms of a coronavirus infection relative to an uninfected state.

In embodiments, the patient is elderly and/or afflicted with one or more comorbidities. In embodiments, the patient has one or more characteristics which provide a high risk for progression to severe disease. In some embodiments, the patient has one or more pre-existing medical conditions that reduce an immune response. In some embodiment, the patient is overweight or obese. In embodiments, the patient has or is receiving one or more agents that render the patient unlikely to have a fulsome immune response to a vaccine. In embodiments, the patient has or is receiving one or more immunosuppressive drugs. In embodiments, the patient is a transplant recipient.

### Viral Vaccines

In embodiments, the patient is vaccinated with a COVID vaccine specific against an infection with SARS-COV2, optionally selected from tozinameran (COMIRNATY) or elasomeran (mRNA-1273, SPIKEVAX), or a variant thereof.

In some embodiments, the COVID vaccine is or comprises an mRNA-based vaccine. In embodiments, the mRNA vaccine comprises one or more non-canonical nucleotides, optionally selected from N1-methylpseudouridine, pseudouridine, and 5-methoxyuridine. In embodiments, the COVID vaccine comprises one or more lipids.

In some embodiments, the COVID vaccine is or comprises a DNA-based vaccine.

In some embodiments, the COVID vaccine is or comprises a viral vector-based vaccine. In embodiments, the viral vector-based vaccine is or comprises an adeno-associated-based vaccine, optionally selected from Ad26.COV2.S or AZD1222 (COVISHIELD, VAXZEVRIA), or a variant thereof.

In some embodiments, the COVID vaccine is or comprises an inactivated vaccine. In other embodiments, the COVID vaccine is or comprises a live-attenuated vaccine. In embodiments, the COVID vaccine is or comprises a subunit, recombinant, polysaccharide, or conjugate vaccine. In some embodiments, the COVID vaccine comprises a spike surface glycoprotein, membrane glycoprotein M, envelope protein E, and/or nucleocapsid phosphoprotein N, or a fragment thereof. In other embodiments, COVID vaccine comprises a nucleic acid encoding a spike surface glycoprotein, membrane glycoprotein M, envelope protein E, and/or nucleocapsid phosphoprotein N, or a fragment thereof.

In other embodiments, the patient is vaccinated with an influenza vaccine. In embodiments, the influenza vaccine is or comprises an mRNA-based vaccine. In some embodiments, the influenza vaccine is or comprises a DNA-based vaccine. In some embodiments, the influenza vaccine is or comprises an inactivated vaccine. In some embodiments, the influenza vaccine is or comprises a live-attenuated vaccine.

In embodiments, the influenza vaccine is or comprises a subunit, recombinant, polysaccharide, or conjugate vaccine. In embodiments, the influenza vaccine comprises an influenza protein, or an antigenic fragment thereof, optionally selected from hemagglutinin (HA) protein, matrix 2 (M2) protein, and neuraminidase, or an antigenic fragment thereof. In other embodiments, the influenza vaccine comprises a nucleic acid encoding an influenza protein, or an antigenic fragment thereof, optionally selected from hemagglutinin (HA) protein, matrix 2 (M2) protein, and neuraminidase, or an antigenic fragment thereof.

### GM-CSF

In embodiments, GM-CSF includes any pharmaceutically safe and effective GM-CSF, or any derivative thereof having the biological activity of GM-CSF. In embodiments, the GM-CSF is recombinant human GM-CSF (rhu GM-CSF), such as sargramostim (LEUKINE). Sargramostim is a biosynthetic, yeast-derived, recombinant human GM-CSF, having of a single 127 amino acid glycoprotein that differs from endogenous human GM-CSF by having a leucine instead of a proline at position 23. Other natural and synthetic GM-CSFs, and derivatives thereof having the biological activity of or substantially similar to natural human GM-CSF, are also useful.

In embodiments, the GM-CSF is produced or producible in bacteria, yeasts, plants, insect cells, and mammalian cells. In embodiments, the GM-CSF is produced or producible in Escherichia coli cells. In embodiments, the GM-CSF is produced or producible in yeast cells. In embodiments, the GM-CSF is produced or producible in Chinese hamster ovary cells (CHO). In embodiments, the GM-CSF is not produced in E. coli cells. In embodiments, the GM-CSF is produced in a cell that allows for glycosylation, e.g., yeast or CHO cells.

In embodiments, the GM-CSF has an amino acid sequence of SEQ ID NO: 1, or a variant of at least about 90%, or at least about 93%, or at least about 95%, or at least about 97%, or at least about 98% identity thereto. In embodiments, the GM-CSF has an amino acid sequence of SEQ ID NO: 2 or SEQ ID NO: 3, or a variant of at least about 90%, or at least about 93%, or at least about 95%, or at least about 97%, or at least about 98% identity thereto. In embodiments, the GM-CSF is one of molgramostim, regramostim and sargramostim. In embodiments, the GM-CSF is sargramostim.

Without wishing to be bound by theory, the core of hGM-CSF consists of four helices that pack at angles. Crystal structures and mutagenic analysis of rhGM-CSF (Rozwarski D A et al., Proteins 26:304-13, 1996) showed that, in addition to apolar side chains in the protein core, 10 buried hydrogen bonding residues involve intramolecular hydrogen bonding to main chain atoms that were better conserved than residues hydrogen bonding to other side chain atoms; 24 solvation sites were observed at equivalent positions in the two molecules in the asymmetric unit, and the strongest among these is located in clefts between secondary structural elements. Two surface clusters of hydrophobic side chains are located near the expected receptor binding regions. Mutagenesis of residues on the helix A/helix C face confirmed the importance of certain Glu, Gly, and Gln residues. These residues are therefore not to be substituted in the functional substitution variants of hGM-CSF for use in the present disclosure and these helices are to be retained in a functional fragments or deletion variants of hGM-CSF for use in this disclosure. Further, in embodiments, one of ordinary skill can reference UniProtKB entry P04141 for structure information to inform the identity of variants.

The N-terminal helix of hGM-CSF governs high affinity binding to its receptor (Shanafelt A B et al., EMBO J 10:4105-12, 1991) Transduction of the biological effects of GM-CSF requires interaction with at least two cell surface receptor components, (one of which is shared with the cytokine IL-5). The above study identified receptor binding determinants in GM-CSF by locating unique receptor binding domains on a series of human-mouse hybrid GM-CSF cytokines. The interaction of GM-CSF with the shared subunit of their high affinity receptor complexes was governed by a very small part of the peptide chains. The presence of a few key residues in the N-terminal α-helix of was sufficient to confer specificity to the interaction.

In embodiments, the amino acid mutations are amino acid substitutions, such as conservative and/or non-conservative substitutions.

“Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices.

As used herein, “non-conservative substitutions” are defined as exchanges of an amino acid by another amino acid listed in a different group of the six standard amino acid groups (1) to (6) shown above.

In embodiments, the substitutions include non-classical amino acids (e.g. selenocysteine, pyrrolysine, N-formylmethionine β-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general).

Modification of the amino acid sequences may be achieved using any known technique in the art e.g., site-directed mutagenesis or PCR-based mutagenesis. Such techniques are described, for example, in Sambrook et al., Molecular Cloning: A Laboratory Manual, Cold Spring Harbor Press, Plainview, N.Y., 1989 and Ausubel et al., Current Protocols in Molecular Biology, John Wiley & Sons, New York, N.Y., 1989.

Without wishing to be bound by theory, the degree of glycosylation of biosynthetic GM-CSFs appears to influence half-life, distribution, and elimination. (Lieschke and Burgess, N. Engl. J. Med. 327:28-35, 1992; Dorr, R. T., Clin. Ther. 15:19-29, 1993; Horgaard et al., Eur. J. Hematol. 50:32-36, 1993). In embodiments, the present GM-CSF molecules are glycosylated.

### Methods of Treatment

In aspects, the present disclosure relates to a method for treating a viral infection in a subject who has received a vaccine to the virus, comprising administering an effective amount of a composition comprising granulocyte-macrophage colony-stimulating factor (GM-CSF) to a subject or patient in need thereof.

In aspects, the present disclosure relates to a method for treating a viral infection in a subject who has received a vaccine to the virus, comprising administering an effective amount of a composition comprising granulocyte-macrophage colony-stimulating factor (GM-CSF) to a subject or patient in need thereof, wherein the patient is characterized by one or more symptoms of the viral infection.

In aspects, the present disclosure provides a method for treating a viral infection in a subject who has received a vaccine to the virus, comprising: (a) selecting a patient who has received a viral vaccine to the virus and has one or more symptoms of the viral infection; and (b) administering with an effective amount of a composition comprising a granulocyte-macrophage colony-stimulating factor (GM-CSF) agent; wherein the effective amount of GM-CSF is sufficient to induce a humoral response and prevent and/or modulate immune reaction to the virus.

In embodiments, the subject or patient has received one or more doses of the vaccine. In some embodiments, the patient has received one dose of the vaccine. In other embodiments, the patient has received two doses of the vaccine. In yet other embodiments, the patient has received three doses of the vaccine.

In embodiments, the patient has received one or more doses of the vaccine at least about two weeks prior to breakthrough infection. In some embodiments, the patient has received one or more booster doses of the vaccine or a variant of the vaccine. In embodiments, the patient has received one or more booster doses of the vaccine or a variant of the vaccine at least about two weeks prior to breakthrough infection. In some embodiments, the patient has received a combination of the vaccines.

In embodiments, the patient has not completed a full prime-boost vaccine regimen (e.g., the patient has received a prime vaccine, but not one or more booster vaccine doses). In embodiments, the patient has had a clinically relevant time gap since their last vaccine dose.

In embodiments, the method causes a significant increase in a humoral response relative to an untreated or pre-treated state. In embodiments, the method causes an increase in an antibody response relative to an untreated or pre-treated state. In embodiments, the method causes a change in the kinetics of antibody response relative to an untreated or pre-treated state. In embodiments, the method causes a change in the magnitude of antibody response relative to an untreated or pre-treated state. In embodiments, the method causes corrects an immune imbalance in the subject.

In embodiments, the method causes an increase in one or more antibody or immunoglobulin isotypes, optionally selected from immunoglobulin M (IgM), immunoglobulin G (IgG), immunoglobulin A (IgA), immunoglobulin D (IgD) and immunoglobulin E (IgE) relative to an untreated or pre-treated state. In embodiments, the method causes an increase in IgM. In embodiments, the method causes an increase in IgG. In other embodiments, the method causes an increase in IgG subclass antibody, IgG4. In embodiments, the method induces immunoglobulin isotype class switching. In embodiments, the method causes an immunoglobulin isotype class switch from IgM isotype to IgG isotype, optionally selected from IgG subclasses IgG1, IgG2, IgG3 and IgG4.

In embodiments, the method causes an immunoglobulin isotype class switch from IgM to IgG4.

In embodiments, the method prevents or mitigates the development or progression of the infection in the patient. In embodiments, the method prevents or mitigates the development or progression of one or more of fever, cough, shortness of breath, diarrhea, upper respiratory symptoms, lower respiratory symptoms, pneumonia, and acute respiratory syndrome.

In embodiments, the method prevents or mitigates the development or progression of one or more of fever, tiredness, dry cough, aches and pains, shortness of breath and other breathing difficulties, diarrhea, upper respiratory symptoms (e.g., sneezing, runny nose, nasal congestion, cough, sore throat), pneumonia, pneumonia respiratory failure, hepatic and renal insufficiency, acute respiratory distress syndrome (ARDS), and a cytokine and an immune imbalance.

In embodiments, the method partially inhibits an immune response to reduce or ablate a cytokine imbalance in the subject. In some embodiments, the method reverses, reduces or prevents a cytokine storm. In some embodiments, the method reverses, reduces or prevents a cytokine storm in the lungs.

In embodiments, the method restores the cytokine imbalance and restores homeostasis in the patient relative to before treatment. In embodiments, the cytokine is one or more of is one or more of IL-6, IL-1, IL-1 receptor antagonist (IL-1ra), IL-2ra, IL-10, IL-18, TNFα, interferon-g (IFN-g), CXCL10, and CCL7. In some embodiments, the cytokine is IL-10. In embodiments, the cytokine is interferon-gamma (IFN-g).

In embodiments, the method reduces or mitigates a likelihood of the patient developing ARDS. In embodiments, the method reduces or mitigates a likelihood of the patient developing lung fibrosis. In embodiments, method reduces or mitigates a likelihood of the patient developing long COVID. In embodiments, the method causes a decrease in viral load in the patient relative to before treatment.

In some embodiments, the method prevents or mitigates the development or progression of the infection that requires hospitalization. In some embodiments, the method prevents or mitigates the development or progression of the infection that causes death of the patient. In other embodiments, the method prevents or mitigates the development or progression of the infection that requires receiving invasive mechanical ventilation. In embodiments, the method prevents or mitigates the development or progression of the infection that requires receiving extracorporeal membrane oxygenation (ECMO). In embodiments, the method prevents or mitigates the development or progression of the infection that requires receiving non-invasive mechanical ventilation. In embodiments, the method prevents or mitigates the development or progression of the infection that requires use of a high flow oxygen device. In embodiments, the method prevents or mitigates the development or progression of the infection that requires receiving supplemental oxygen. In embodiments, the method prevents or mitigates the development or progression of the infection that requires receiving ongoing medical care.

In embodiments, the method prevents or mitigates the development or progression of the infection to a NIAID Ordinal Scale score of 3 to 8. In some embodiments, the method prevents or mitigates the development or progression of the infection to an NIAID Ordinal Scale score of 8. In some embodiments, the method prevents or mitigates the development or progression of the infection to an NIAID Ordinal Scale score of 7. In some embodiments, the method prevents or mitigates the development or progression of the infection to an NIAID Ordinal Scale score of 6. In some embodiments, the method prevents or mitigates the development or progression of the infection to an NIAID Ordinal Scale score of 5. In some embodiments, the method prevents or mitigates the development or progression of the infection to a NIAID Ordinal Scale score of 4. In some embodiments, the method prevents or mitigates the development or progression of the infection to a NIAID Ordinal Scale score of 3.

In embodiments, the method prevents or mitigates the development or progression of the infection as measured by patient global assessment scores (e.g., Clinical Global Impression (CGI) scale). In embodiments, the method improves symptoms and functional abilities in a patient as measured by patient global assessment scores (e.g., Clinical Global Impression (CGI) scale).

### Pharmaceutically Acceptable Salts and Excipients

The compositions described herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

Pharmaceutically acceptable salts include, by way of non-limiting example, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, pamoate, phenylacetate, trifluoroacetate, acrylate, chlorobenzoate, dinitrobenzoate, hydroxybenzoate, methoxybenzoate, methylbenzoate, o-acetoxybenzoate, naphthalene-2-benzoate, isobutyrate, phenylbutyrate, α-hydroxybutyrate, butyne-1,4-dicarboxylate, hexyne-1,4-dicarboxylate, caprate, caprylate, cinnamate, glycollate, heptanoate, hippurate, malate, hydroxymaleate, malonate, mandelate, mesylate, nicotinate, phthalate, teraphthalate, propiolate, propionate, phenylpropionate, sebacate, suberate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, xylenesulfonate, and tartarate salts.

The term “pharmaceutically acceptable salt” also refers to a salt of the compositions of the present disclosure having an acidic functional group, such as a carboxylic acid functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N, N-dimethyl-N-(2-hydroxyethyl)amine or tri-(2-hydroxyethyl)amine; N-methyl-D-glucamine; and amino acids such as arginine, lysine, and the like.

In embodiments, the compositions described herein are in the form of a pharmaceutically acceptable salt.

### Pharmaceutical Compositions and Formulations

In embodiments, the present disclosure pertains to pharmaceutical compositions comprising the compositions, e.g., GM-CSF and/or an additional therapeutic agent, described herein and a pharmaceutically acceptable carrier or excipient. Any pharmaceutical compositions described herein can be administered to a subject as a component of a composition that comprises a pharmaceutically acceptable carrier or vehicle. Such compositions can optionally comprise a suitable amount of a pharmaceutically acceptable excipient so as to provide the form for proper administration.

In embodiments, pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In embodiments, the pharmaceutically acceptable excipients are sterile when administered to a subject. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference.

The present disclosure includes the described pharmaceutical compositions (and/or additional therapeutic agents) in various formulations. Any pharmaceutical composition (and/or additional therapeutic agents) described herein can take the form of solutions, suspensions, emulsion, drops, tablets, pills, pellets, capsules, capsules containing liquids, gelatin capsules, powders, sustained-release formulations, suppositories, emulsions, aerosols, sprays, suspensions, lyophilized powder, frozen suspension, desiccated powder, or any other form suitable for use. In embodiments, the composition is in the form of a capsule. In embodiments, the composition is in the form of a tablet. In embodiments, the pharmaceutical composition is formulated in the form of a soft-gel capsule. In a further embodiment, the pharmaceutical composition is formulated in the form of a gelatin capsule. In embodiments, the pharmaceutical composition is formulated as a liquid.

Where necessary, the present pharmaceutical compositions (and/or additional therapeutic agents) can also include a solubilizing agent. Also, the agents can be delivered with a suitable vehicle or delivery device as known in the art. Combination therapies outlined herein can be co-delivered in a single delivery vehicle or delivery device.

The formulations comprising the present pharmaceutical compositions (and/or additional therapeutic agents) of the present disclosure may conveniently be presented in unit dosage forms and may be prepared by any of the methods well known in the art of pharmacy. Such methods generally include the step of bringing the therapeutic agents into association with a carrier, which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In embodiments, any pharmaceutical compositions (and/or additional therapeutic agents) described herein is formulated in accordance with routine procedures as a composition adapted for a mode of administration described herein.

Routes of administration include, for example: oral, intradermal, intramuscular, intraperitoneal, intravenous, subcutaneous, intranasal, epidural, sublingual, intranasal, intracerebral, intravaginal, transdermal, rectally, by inhalation, or topically. Administration can be local or systemic. In embodiments, the administering is affected orally. In embodiments, the administration is by parenteral injection. The mode of administration can be left to the discretion of the practitioner and depends in-part upon the site of the medical condition. In most instances, administration results in the release of any agent described herein into the bloodstream.

In embodiments, the GM-CSF (and/or other additional therapeutic agents) is administered via a subcutaneous route.

In embodiments, the GM-CSF (and/or other additional therapeutic agents) is administered via an oral (e.g., sublingual) route.

In embodiments, the GM-CSF (and/or additional therapeutic agents) is administered via an intranasal route. In embodiments, the GM-CSF (and/or additional therapeutic agents) is administered by inhalation. Without wishing to be bound by theory, inhalation of sargramostim produces low systemic exposure in the patient.

In embodiments, the GM-CSF (and/or additional therapeutic agents) is administered to the lung.

In embodiments, the GM-CSF (and/or additional therapeutic agents) is administered via aerosol or nebulizer.

In embodiments, the aerosol or nebulizer is selected from liquid nebulization, dry powder dispersion and meter-dose administration. In embodiments, the aerosol or nebulizer is selected from jet flow or mesh vibrating.

In embodiments, the GM-CSF (and/or additional therapeutic agents) is administered by inhalation and mediates local and/or peripheral cellular responses. For instance, in embodiments, the GM-CSF (and/or additional therapeutic agents) is administered by inhalation and mediates an increase in lymphocytes and/or eosinophils in the peripheral blood.

Accordingly, in embodiments, there is provided a method of modulating peripheral immune cells to cause an anti-infective effect through local administration (e.g., inhalation).

In embodiments, the pharmaceutical compositions (and/or additional therapeutic agents) described herein are formulated in accordance with routine procedures as a composition adapted for oral administration. Compositions for oral delivery can be in the form of tablets, lozenges, aqueous or oily suspensions, granules, powders, emulsions, capsules, syrups, or elixirs, for example. Orally administered compositions can comprise one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving any pharmaceutical compositions (and/or additional therapeutic agents) described herein are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be useful. Oral compositions can include standard excipients such as mannitol, lactose, starch, magnesium stearate, sodium saccharin, cellulose, and magnesium carbonate. In embodiments, the excipients are of pharmaceutical grade. Suspensions, in addition to the active compounds, may contain suspending agents such as, for example, ethoxylated isostearyl alcohols, polyoxyethylene sorbitol and sorbitan esters, microcrystalline cellulose, aluminum metahydroxide, bentonite, agar-agar, tragacanth, etc., and mixtures thereof.

Dosage forms suitable for parenteral administration (e.g., intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g., lyophilized composition), which can be dissolved or suspended in sterile injectable medium immediately before use. They may contain, for example, suspending or dispersing agents known in the art. Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, NJ) or phosphate buffered saline (PBS). The carrier should be stable under the conditions of manufacture and storage and should be preserved against microorganisms. The carrier can be a solvent or dispersion medium containing, for example, water, ethanol, polyol (for example, glycerol, propylene glycol, and liquid polyetheylene glycol), and suitable mixtures thereof.

The compositions provided herein, alone or in combination with other suitable components, can be made into aerosol formulations (i.e., “nebulized”) to be administered via inhalation. Aerosol formulations can be placed into pressurized acceptable propellants, such as dichlorodifluoromethane, propane, nitrogen, and the like.

Any pharmaceutical compositions (and/or additional therapeutic agents) described herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropyl cellulose, hydropropylmethyl cellulose, polyvinylpyrrolidone, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the agents described herein. The disclosure thus provides single unit dosage forms suitable for oral administration such as, but not limited to, tablets, capsules, gelcaps, and caplets that are adapted for controlled- or sustained-release.

Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In embodiments, a controlled-release system can be placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

Pharmaceutical formulations preferably are sterile. Sterilization can be accomplished, for example, by filtration through sterile filtration membranes. Where the composition is lyophilized, filter sterilization can be conducted prior to or following lyophilization and reconstitution.

### Administration and Dosage

It will be appreciated that the actual dose of the composition to be administered according to the present disclosure will vary according to the particular dosage form, and the mode of administration. Many factors that may modify the action of the composition (e.g., body weight, gender, diet, time of administration, route of administration, rate of excretion, condition of the subject, drug combinations, genetic disposition and reaction sensitivities) can be taken into account by those skilled in the art. Administration can be carried out continuously or in one or more discrete doses within the maximum tolerated dose. Optimal administration rates for a given set of conditions can be ascertained by those skilled in the art using conventional dosage administration tests.

In embodiments, the GM-CSF is administered at a total dose of about 125 μg, about 150 μg, or about 200 μg, or about 250 μg, or about 300 μg, or about 350 μg. In embodiments, the GM-CSF is administered at a dose of about 125 μg, about 150 μg, or about 200 μg, or about 250 μg, or about 300 μg, or about 350 μg, once daily. In embodiments, the GM-CSF is administered at a total dose of about 250 μg, once daily.

In embodiments, the GM-CSF is administered at a dose of about 125 μg, about 150 μg, or about 200 μg, or about 250 μg, or about 300 μg, or about 350 μg, once daily. In embodiments, the GM-CSF is sargramostim, administered at a dose of about 250 μg, once daily. In embodiments, the GM-CSF is sargramostim, administered at a dose of about 250 μg, once daily, for at least 5 consecutive days.

In embodiments, the GM-CSF is sargramostim, administered at a dose of about 250 μg, once daily, for at least 5 consecutive days following a positive test for a viral infection or the start of symptoms of the viral infection. In embodiments, the GM-CSF is sargramostim, administered at a dose of about 250 μg, once daily, for 5 consecutive days following a positive test for COVID-19.

### Combination Therapy and Additional Therapeutic Agents

In embodiments of the disclosure “treating” includes administration of the present combination to an individual who is at risk for potential exposure, or who has been exposed to or a source of the virus. “Treating” means, for example, administration of a viral vaccine (e.g., COVID vaccine or influenza vaccine or another vaccine for a virus) along with GM-CSF, including the combination of the present disclosure, to an individual who tested positive for the viral infection but has or has not yet shown mild or moderate symptoms, and administration of a combination of the present disclosure to an individual who shows or has shown viral infection symptoms.

In embodiments, the pharmaceutical composition of the present disclosure is co-administered in conjunction with additional therapeutic agent(s). Co-administration can be simultaneous or sequential.

In embodiments, the additional therapeutic agent and the GM-CSF of the present disclosure are administered to a subject simultaneously. The term “simultaneously” as used herein, means that the additional therapeutic agent and the GM-CSF are administered with a time separation of no more than about 60 minutes, such as no more than about 30 minutes, no more than about 20 minutes, no more than about 10 minutes, no more than about 5 minutes, or no more than about 1 minute. Administration of the additional therapeutic agent and the GM-CSF can be by simultaneous administration of a single formulation (e.g., a formulation comprising the additional therapeutic agent and the GM-CSF composition) or of separate formulations (e.g., a first formulation including the additional therapeutic agent and a second formulation including the GM-CSF composition).

Co-administration does not require the therapeutic agents to be administered simultaneously, if the timing of their administration is such that the pharmacological activities of the additional therapeutic agent and the GM-CSF overlap in time, thereby exerting a combined therapeutic effect. For example, the additional therapeutic agent and the targeting moiety, the GM-CSF composition can be administered sequentially. The term “sequentially” as used herein means that the additional therapeutic agent and the GM-CSF are administered with a time separation of more than about 60 minutes. For example, the time between the sequential administration of the additional therapeutic agent and the GM-CSF can be more than about 60 minutes, more than about 2 hours, more than about 5 hours, more than about 10 hours, more than about 1 day, more than about 2 days, more than about 3 days, more than about 1 week apart, more than about 2 weeks apart, or more than about one month apart. The optimal administration times will depend on the rates of metabolism, excretion, and/or the pharmacodynamic activity of the additional therapeutic agent and the GM-CSF being administered. Either the additional therapeutic agent or the GM-CSF composition may be administered first.

Co-administration also does not require the therapeutic agents to be administered to the subject by the same route of administration. Rather, each therapeutic agent can be administered by any appropriate route, for example, parenterally or non-parenterally.

In embodiments, the GM-CSF described herein acts synergistically when co-administered with another therapeutic agent. In embodiments, the targeting moiety, the GM-CSF composition and the additional therapeutic agent may be administered at doses that are lower than the doses employed when the agents are used in the context of monotherapy.

In embodiments, the additional therapeutic agent is selected from nirmatrelvir/ritonavir, remdesivir; favipiravir; galidesivir; prezcobix; lopinavir; and/or ritonavir; and/or arbidol lopinavir/ritonavir; and/or ribavirin; and/or IFN-beta; xiyanping; anti-VEGF-A; fingolimod; carrimycin; hydroxychloroquine; darunavir and cobicistat; methylprednisolone; brilacidin; leronlimab; thalidomide, bamlanivimab, casirivimab, and imdevimab.

In embodiments, the additional therapeutic agent is nirmatrelvir/ritonavir (PAXLOVID). In embodiments, the additional therapeutic agent is an antibody directed against an antigen of the virus. In embodiments, the additional therapeutic agent is a monoclonal antibody directed against a coronavirus antigen. In some embodiments, the additional therapeutic agent is a steroid, optionally selected from dexamethasone or prednisone.

In embodiments, the additional therapeutic agent is selected from pill, liquids, nasal sprays and/or drops, throat sprays and/or drops, and eye drops.

In some embodiments, the method prevents or mitigates the development or progression of the infection that requires receiving an additional therapeutic agent.

### Methods of Detecting Therapeutic Effect

In embodiments, there is provided a method for detecting the likelihood of successful treatment or response to the present treatment for a viral infection in a patient. In embodiments, there is provided a method for detecting the likelihood of successful treatment or response to treatment with the present disclosure for a viral infection in a patient or subject. In embodiments, there is provided a method for detecting the likelihood of successful treatment or response and/or clinical benefit to the present treatment for a viral infection in a patient based in the improvement in patient global assessment scores (e.g., Clinical Global Impression (CGI) scale) or NIAID Ordinal Scale score.

In embodiments, the breakthrough viral infection is assayed or assayable in a biological sample from the patient (e.g., blood, respiratory fluid (e.g., from an oropharyngeal (OP) or nasopharyngeal (NP) swab, sputum, a nasal secretion), saliva or stool.

In embodiment, the breakthrough viral infection is assayed or assayable using a measurement of a viral nucleic acid or protein. In some the breakthrough viral infection is assayed or assayable using PCR. In other embodiments, the breakthrough viral infection is assayed or assayable using a rapid test. In embodiments, the breakthrough viral infection is assayed or assayable using a lateral flow antigen test.

In embodiments, the present disclosure includes monitoring the viral load during the course of treatment. In embodiments, the present disclosure causes a decrease in the viral load in the patient relative to an untreated or pre-treated state. In embodiment, the monitoring of the viral load is assayed or assayable using a measurement of a viral nucleic acid or protein. In some the monitoring of the viral load is assayed or assayable using PCR. In other embodiments, the monitoring of the viral load is assayed or assayable using a rapid test. In embodiments, the monitoring of the viral load is monitoring of viral load is assayed or assayable using a lateral flow antigen test.

In embodiments, the present disclosure restores the immune imbalance and/or restores homeostasis in the patient relative to an untreated or pre-treated state.

In embodiments, the present disclosure causes a decrease in viral symptoms in the patient, and therefore this reduction is used as a biomarker for treatment. In embodiments, the present disclosure causes a decrease in the amount of virus in a patient, and therefore this reduction is used as a biomarker for treatment. For instance, a decrease in viral load and/or viral symptoms directs towards discontinuation of administration of GM-CSF or a reduction in dosage of GM-CSF.

In embodiments, a worsening of symptoms in the patient with a viral infection at any timepoint directs toward continued administration of the GM-CSF. In embodiments, a worsening in the NIAID Ordinal Scale score in a patient with a viral infection at any timepoint directs toward continued administration of GM-CSF.

In embodiments, a decrease in a viral load and/or viral symptoms directs towards discontinuation of administration of GM-CSF or a reduction in dosage of GM-CSF. In embodiments, a worsening in the NIAID Ordinal Scale score in a patient with a viral infection at any timepoint directs toward continued administration of GM-CSF.

In embodiments, the method prevents or mitigates the development or progression of the viral infection that requires receiving an additional therapeutic agent. In embodiments, an improvement in patient global assessment scores (e.g., Clinical Global Impression (CGI) scale) in a patient with a viral infection at any timepoint directs discontinuation of administration of GM-CSF a reduction in dosage of GM-CSF.

### Uses of Molgramostim or Regramostim

In embodiments, the present disclosure pertains to the use of molgramostim or regramostim in a method of treating or preventing a viral infection and/or viral symptoms, by administering via inhalation and, optionally, modulating an immune response in the patient.

In embodiments, such molgramostim is used in a method of treating or ameliorating an immune response to a specific viral infection. In embodiments, such regramostim is used in a method of treating or preventing an immune response to a specific viral infection.

### Sequences

SEQ ID NO: 1 is wild type GM-CSF:
APARSPSPSTQPWEHVNAIQEAPRLLNLSRDTAAEMNETVEVISEMFDLQ
EPTCLQTRLELYKQGLRGSLTKLKGPLTMMASHYKQHCPPTPETSCATQI
ITFESFKENLKDFLLVIPFDCWEPVQE.
SEQ ID NO: 2 is sargramostim:
APARSPSPSTQPWEHVNAIQEALRLLNLSRDTAAEMNETVEVISEMFDLQ
EPTCLQTRLELYKQGLRGSLTKLKGPLTMMASHYKQHCPPTPETSCATQI
ITFESFKENLKDFLLVIPFDCWEPVQE.
SEQ ID NO: 3 is molgramostim:
APARSPSPSTQPWEHVNAIQEARRLLNLSRDTAAEMNETVEVISEMFDLQ
EPTCLQTRLELYKQGLRGSLTKLKGPLTMMASHYKQHCPPTPETSCATQI
ITFESFKENLKDFLLVIPFDCWEPVQE.

### Definitions

The following definitions are used in connection with the present disclosure. Unless defined otherwise, all technical and scientific terms used herein have the same meaning as commonly understood to one of skill in the art to which this disclosure belongs.

An “effective amount,” when used in connection with an agent effective for the treatment of a viral infection is an amount that is effective for treating or mitigating a response to a virus.

As used herein, “a,” “an,” or “the” can mean one or more than one. Further, the term “about” when used in connection with a referenced numeric indication means the referenced numeric indication plus or minus up to 10% of that referenced numeric indication. For example, the language “about 50” covers the range of 45 to 55.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the disclosure, the present disclosure, or embodiments thereof, may alternatively be described using alternative terms such as “consisting of” or “consisting essentially of.”

This disclosure is further illustrated by the following non-limiting examples.

### EXAMPLES

### Example 1: Design of Clinical Trial of Sargramostim (LEUKINE) for Treatment of COVID-19

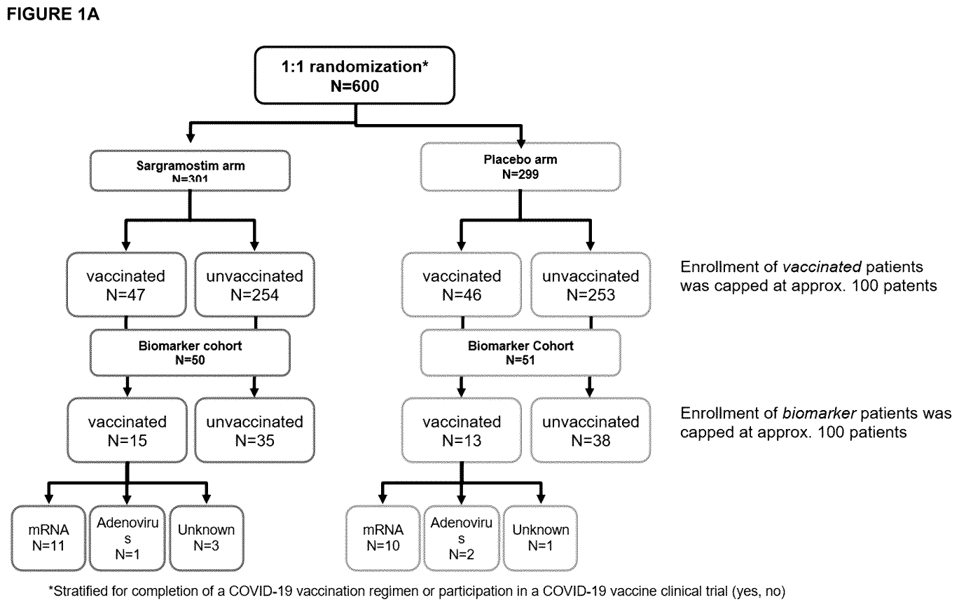

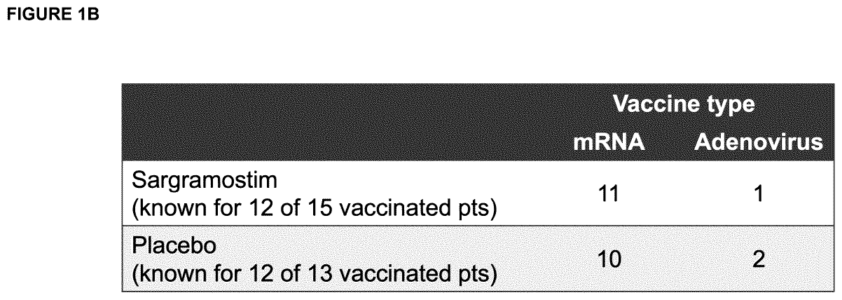

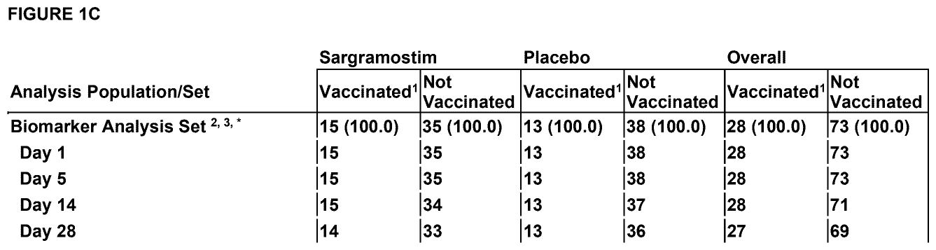

A prospective, randomized, open-label, interventional study to investigate the efficacy of sargramostim (LEUKINE) in vaccinated and unvaccinated patient who tested positive for COVID-19 patients was conducted (FIG. 1A). 600 patients enrolled in the clinical trial had mild to moderate COVID-19 and were not hospitalized. Most patients in the study (82.5%) were unvaccinated for COVID-19 or had not participated in a COVID-19 vaccination trial. There was no significant difference between treatment groups in the percentage of vaccinated patients. Treatment groups were well-balanced for mean age, sex, race, ethnicity and body mass index (BMI). The Biomarker Cohort patients were comparable to the overall study population in terms of demographic and baseline disease characteristics. In the Biomarker Cohort patients, 28% of were vaccinated for COVID-19 or had participated in a COVID-19 vaccination trial (FIG. 1B). Of these vaccinated Biomarker Cohort patients, most had been vaccinated with an mRNA COVID-19 vaccine (sargramostim-treated=11, placebo-treated=10) (FIG. 1C).

### Example 2: Clinical Results

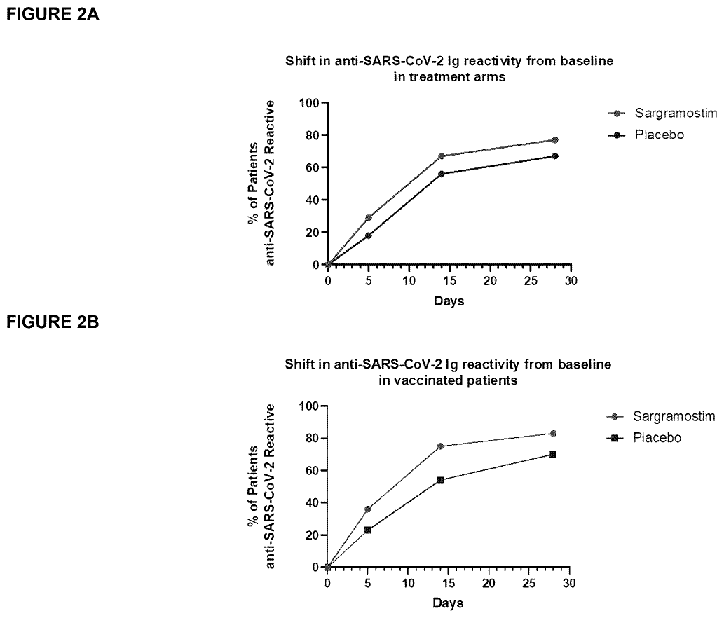

The generation of antibody production against SARS-COV-2 was evaluated using a qualitative assessment of Pan-Ig responses against the SARS-COV-2 virus by using the Roche's Elecsys Total Anti-SARS-COV-2 test on Cobas e801. The Elecsys Anti-SARS-COV-2 assay is an electrochemiluminescence immunoassay (ECLIA) utilizing a recombinant protein representing the nucleocapsid (N) antigen for the determination of antibodies against SARS-COV-2. As shown in FIG. 2A, overall, a higher percentage of sargramostim treated patients turned reactive for anti-SARS-COV-2 serum Ig compared to placebo treated patients at Day 5 (29% vs 18%), Day 14 (67% vs 56%) and Day 28 (77% vs 67%) respectively. More vaccinated sargramostim treated patients turned reactive for anti-SARS-COV-2 serum Ig compared to placebo at Day 5 (36% vs 23%), Day 14 (75% vs 54%) and Day 28 (83% vs 69%) respectively (FIG. 2B).

A panel of up to 10 SARS COV2 antigens were tested in the two biophysical (Luminex) assays in the Isotyping & Subclassing assays to determine patient responses. Antigen-specific antibody isotyping/subclassing was analyzed using the fluorescently coded microspheres to capture up to 500 antigen specificities simultaneously and profile the isotype/subclass distribution in an antigen-specific manner. The target antigens, as well as a positive control antigen (influenza H1 (A/California/7/2009)) and a negative control antigen (Ebola virus GP), were covalently coupled to beads via primary amine conjugation or via biotin: streptavidin interactions. The beads are incubated with diluted serum, allowing “on-bead” affinity purification of antigen-specific antibodies. The bound antigen-specific antibodies are subsequently probed with antibodies detecting IgG, IgG1, IgG2, IgG3, IgG4, IgA1, IgA2, and IgM. The data is reported as the median fluorescence intensity of phycoerythrin (PE) for a specific bead channel. Each sample is run in biological duplicate, and the mean value of the duplicates is reported (FIGS. 2A and 2B).

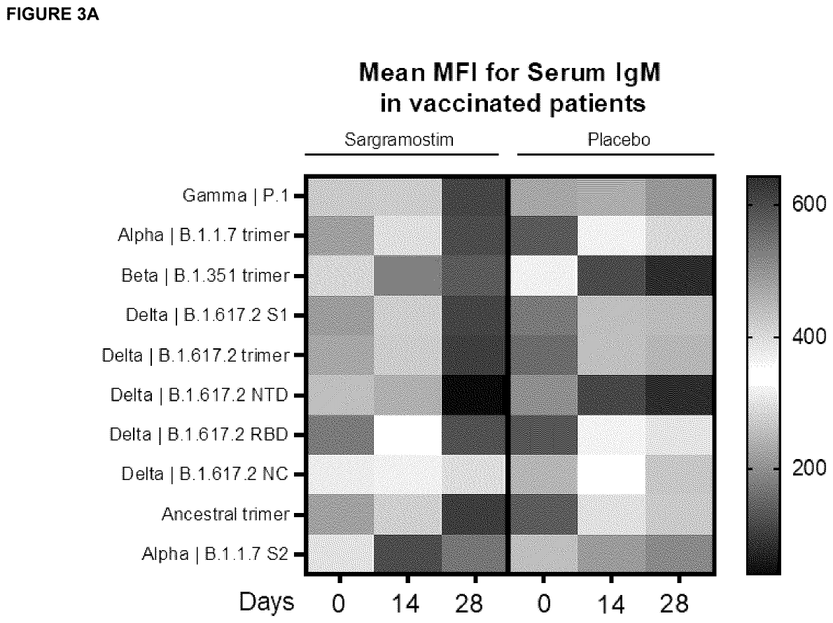

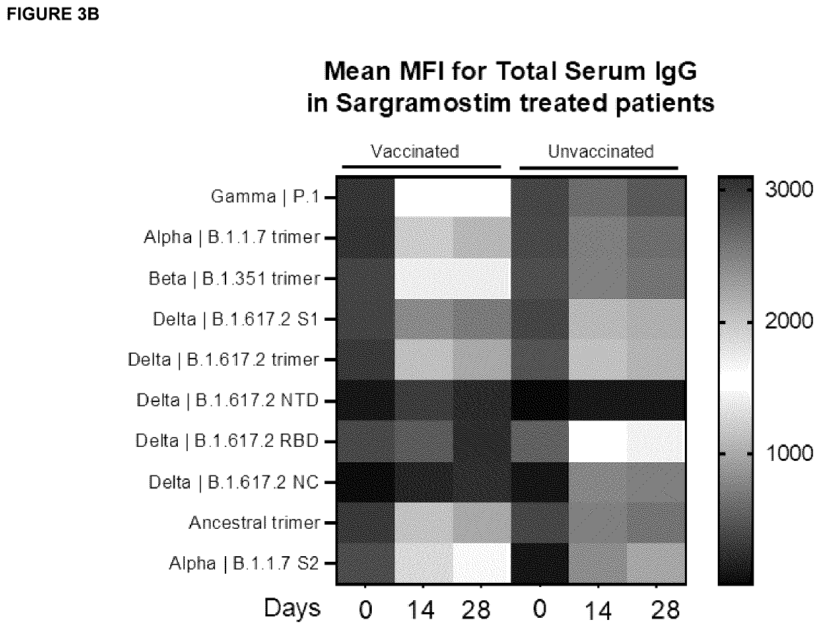

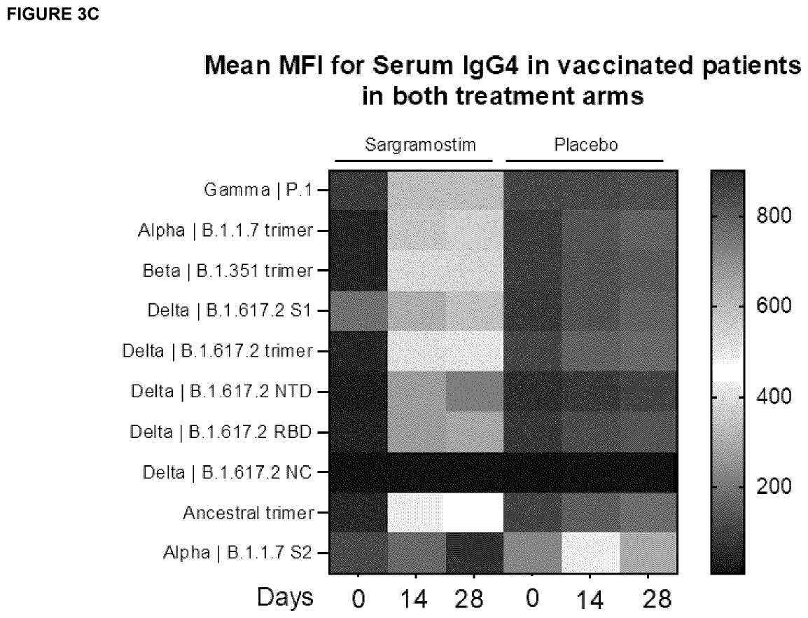

Kinetics and magnitude of antibody responses against SARS-COV-2 antigens differed by group. After an initial IgM-titer peak at day 14, IgM titers at day 28 were lower in the vaccinated-sargramostim arm than the vaccinated-placebo arm (data on IgM, FIG. 3A). Although IgG titers were similar between treatment arms, overall titers were higher for vaccinated than unvaccinated patients across both the sargramostim (data on total IgG, FIG. 3B) and placebo arms (data not shown). The vaccinated-sargramostim arm had higher IgG4 titers associated with IgG4-isotype-class switching than the vaccinated-placebo arm (data on IgG4, FIG. 3C).

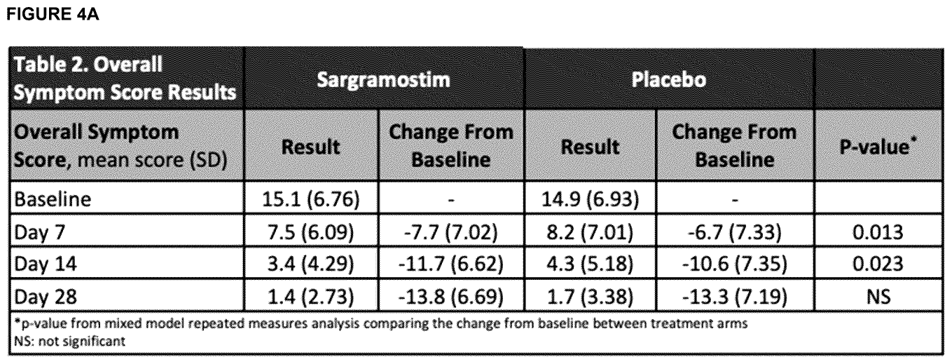

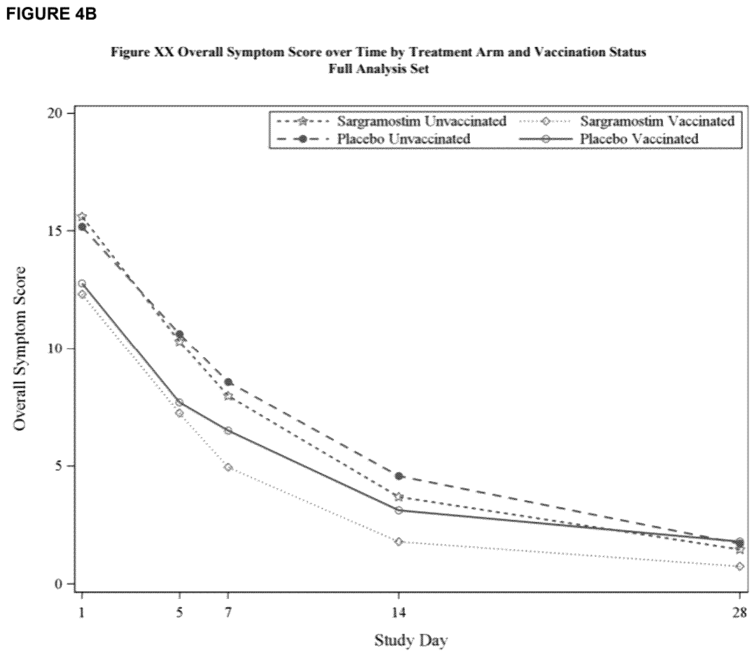

Symptoms were evaluated and scored in all (vaccinated and unvaccinated) patients who received sargramostim vs. placebo. Enrollment of COVID-vaccinated patients was limited to 100 patients and most had received a mRNA COVID vaccine. To evaluate the change in symptoms during the study, patients completed an electronic symptom score questionnaire at approximately the same time every day up to day 28. The questionnaire assessed 14 common symptoms of COVID-19 (i.e., cough, headache, body aches, etc.) with patients rating each symptom over the prior 24 hours (0=none, 1=mild, 2=moderate, 3=severe). The overall symptom score was calculated as a sum of the individual scores from the 14 questions each day. Data analyzed at day 7, 14, and day 28. Importantly, the sargramostim cohort had a greater reduction in overall symptom score than the placebo cohort (FIG. 4A) regardless of vaccination status. This effect was more pronounced in the vaccinated-sargramostim cohort/arm and all vaccinated patients had a lower overall symptom score at baseline than unvaccinated patients (FIG. 4B). Vaccinated patients also had a lower baseline overall symptom score. The sargramostim arm had a greater reduction in overall symptom score than the placebo arm over 28 days. The effect of sargramostim on patients' overall symptom score is observed from day 7 onwards.

Random forest classifier models were designed to discriminate treatment and vaccination status and random forest regressors models were designed to map humoral features to viral load. The performance of the models was validated using a rigorous k-fold cross-validation and permutation testing framework. Cross-validation is a widely used strategy in machine learning to evaluate the performance of the model with data held out, and during k-fold cross-validation, the models were built using subsets of the data and then tested on the remainder of the data to gain an unbiased estimate of model performance. Permutation testing generates suitable “negative control” models in a matched cross-validation framework to assess the significance of the observed model performance. Feature importance estimates were established by permutation of out-of-bag predictor observations within each fold. In essence, without wishing to be bound by theory, the importance of a feature corresponds to the drop in accuracy when the information of that feature is removed from the model.

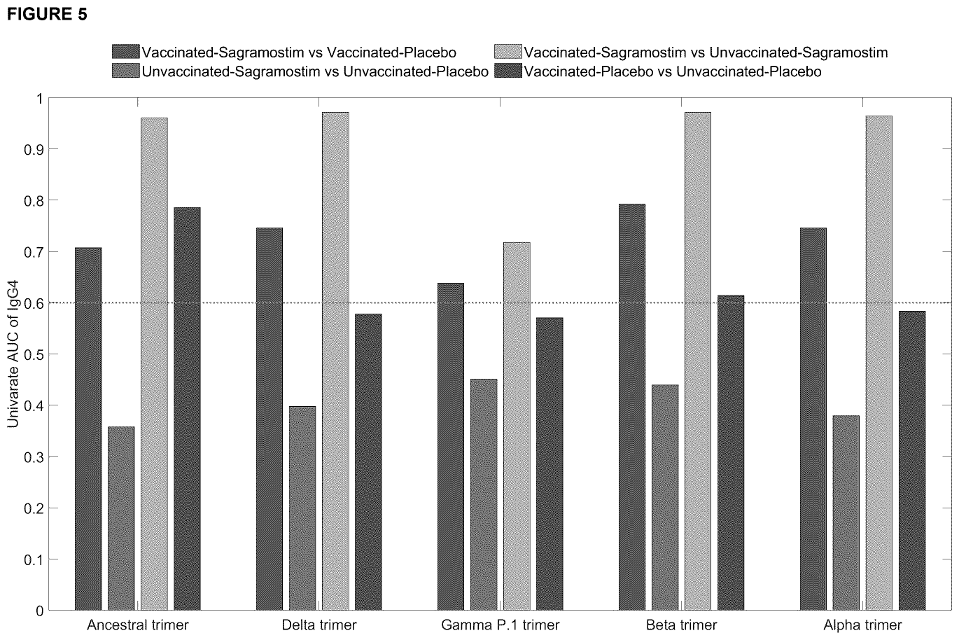

Computational models of sargramostim and vaccination status highlight, inter alia, that IgG4 is important in a synergistic immune response against COVID-specific antigens. Serum IgG4 antibody levels were assessed in 99 patients of the biomarker cohort (Sargramostim n=48; Placebo n=51). As shown in FIG. 5, Sargramostim enhances IgG4 antibody profile (shown in AUC) in vaccinated patients. For each trimer, enrichment by Sargramostim to IgG4 is enhanced by vaccination (compare third and fourth bars, i.e., “Unvaccinated-Sagramostim vs Unvaccinated-Placebo” and “Vaccinated-Sagramostim vs Vaccinated-Placebo”.). For each trimer, enrichment by vaccination to IgG4 is enhanced by Sargramostim (compare first and second bars, i.e., “Vaccinated-Placebo vs Unvaccinated-Placebo” and “Vaccinated-Sagramostim vs Unvaccinated-Sagramostim”) (FIG. 5). This pair of enrichments indicates that there is underlying synergy between vaccination and Sargramostim. Note that the area under the curve (AUC) is a measure of the association between two subsections of the cohort, vaccinated-sargramostim versus vaccinated-placebo. An AUC>0.6 indicates that IgG4 concentration was higher in the vaccinated-sargramostim arm whereas an AUC <0.4 indicates that either feature was higher in the vaccinated-placebo arm. AUCs between 0.4 and 0.6 are indicative of no difference.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

As used herein, all headings are simply for organization and are not intended to limit the disclosure in any manner. The content of any individual section may be equally applicable to all sections.

What is claimed is:

1. A method of treating or preventing a breakthrough viral infection, comprising:
(a) selecting a patient who has received a vaccination to the infection and has one or more symptoms of the viral infection; and
(b) administering with an effective amount of a granulocyte-macrophage colony-stimulating factor (GM-CSF) agent;
wherein the effective amount of GM-CSF is sufficient to modulate humoral immunity in the patient.

2. A method for treating a vaccinated patient with a breakthrough viral infection, administering an effective amount of a composition comprising a granulocyte-macrophage colony-stimulating factor (GM-CSF) agent,
wherein the effective amount is sufficient to induce a significant humoral response and a reduction of the viral load and/or viral infection symptoms.

3. The method of claim 1 or claim 2, wherein the breakthrough viral infection is a respiratory viral infection.

4. The method of any one of claims 1 to 3, wherein the breakthrough viral infection is an infection with a coronavirus.

5. The method of claim 4, wherein the coronavirus is selected from
(i) a betacoronavirus, optionally selected from severe acute respiratory syndrome coronavirus 2 (SARS-COV-2), SARS-COV, Middle East respiratory syndrome-corona virus (MERS-COV), HCoV-HKU1, and HCoV-OC43; and
(ii) an alphacoronavirus, optionally selected from HCoV-NL63 and HCoV-229E.

6. The method of claim 4 or claim 5, wherein the coronavirus is SARS-COV-2 and/or the patient is afflicted with COVID-19.

7. The method of claim 5 or claim 6, wherein the SARS-COV-2 is a variant of SARS-CoV-2.

8. The method of claim 7, wherein the variant of SARS-COV-2 is selcted from alpha (B.1.1.7), beta (B.1.351), gamma (P.1), delta (B.1.617.2), epsilon (B.1.427 or B.1.429), eta (B.1.525), iota (B.1.526), kappa (B.1.617.1), lambda (C.37), mu (B.1.621 or B.1.621.1), omicron (BA.1, B.1.1.529, BA.2, BA.2.75, BA.3, BA.4/BA.5, BN.1BQ.1, XBB, XBB.1.5, XAK, XAY, XBC), and zeta (P.2).

9. The method of any one of claims 1 to 8, wherein the patient is vaccinated with a COVID vaccine.

10. The method of claim 9, wherein the COVID vaccine is or comprises an mRNA-based vaccine.

11. The method of claim 10, wherein the mRNA vaccine comprises one or more non-canonical nucleotides.

12. The method of claim 11, wherein the non-canonical nucleotides are selected from N1-methylpseudouridine, pseudouridine, and 5-methoxyuridine.

13. The method of any one of claims 9 to 12, wherein the COVID vaccine comprises one or more lipids.

14. The method of any one of claims 9 to 13, wherein the COVID vaccine is tozinameran (COMIRNATY) or elasomeran (mRNA-1273, SPIKEVAX), or a variant thereof.

15. The method of claim 9, wherein the COVID vaccine is or comprises a DNA-based vaccine.

16. The method of claim 15, wherein the COVID vaccine is or comprises a viral vector-based vaccine.

17. The method of claim 16, wherein the viral vector-based vaccine is or comprises an adeno-associated-based vaccine.

18. The method of any one of claims 15 to 17, wherein the vaccine is Ad26.COV2.S or AZD1222 (COVISHIELD, VAXZEVRIA), or a variant thereof.

19. The method of claim 9, wherein the COVID vaccine is or comprises an inactivated vaccine.

20. The method of claim 19, wherein the COVID vaccine is or comprises a live-attenuated vaccine.

21. The method of claim 9, wherein the COVID vaccine is or comprises a subunit, recombinant, polysaccharide, or conjugate vaccine.

22. The method of claim 21, wherein the COVID vaccine comprises a spike surface glycoprotein, membrane glycoprotein M, envelope protein E, and/or nucleocapsid phosphoprotein N, or a fragment thereof.

23. The method of claim 21, wherein the COVID vaccine comprises a nucleic acid encoding a spike surface glycoprotein, membrane glycoprotein M, envelope protein E, and/or nucleocapsid phosphoprotein N, or a fragment thereof.

24. The method of any one of claims 1 to 3, wherein the breakthrough viral infection is an infection with an influenza virus.

25. The method of claim 24, wherein the influenza virus is selected from Type A, Type B, Type C, and Type D influenza.

26. The method of claim 24 or claim 25, wherein the breakthrough viral infection is pandemic 2009 influenza A (H1N1) or avian influenza A (H5N1).

27. The method of any one of claims 24 to 26, wherein the patient is vaccinated with an influenza vaccine.

28. The method of claim 27, wherein the influenza vaccine is or comprises an mRNA-based vaccine.

29. The method of claim 27, wherein the influenza vaccine is or comprises a DNA-based vaccine.

30. The method of claim 27, wherein the influenza vaccine is or comprises an inactivated vaccine.

31. The method of claim 27, wherein the influenza vaccine is or comprises a live-attenuated vaccine.

32. The method of claim 27, wherein the influenza vaccine is or comprises a subunit, recombinant, polysaccharide, or conjugate vaccine.

33. The method of claim 32, wherein the influenza vaccine comprises an influenza protein, or an antigenic fragment thereof, optionally selected from hemagglutinin (HA) protein, matrix 2 (M2) protein, and neuraminidase, or an antigenic fragment thereof.

34. The method of claim 32, wherein the influenza vaccine comprises a nucleic acid encoding an influenza protein, or an antigenic fragment thereof, optionally selected from hemagglutinin (HA) protein, matrix 2 (M2) protein, and neuraminidase, or an antigenic fragment thereof.

35. The method of any one of claims 1 to 34, wherein the patient has received one or more doses of the vaccine.

36. The method of claim 35, wherein the patient has received one dose of the vaccine.

37. The method of claim 35, wherein the patient has received two doses of the vaccine.

38. The method of claim 35, wherein the patient has received three doses of the vaccine.

39. The method of claim 35, wherein the patient has received one or more doses of the vaccine at least about two weeks prior to breakthrough infection.

40. The method of any one of claims 1 to 34, wherein the patient has received one or more booster doses of the vaccine or a variant of the vaccine.

41. The method of claim 40, wherein the patient has received one or more booster doses of the vaccine or a variant of the vaccine at least about two weeks prior to breakthrough infection.

42. The method of any one of the claims 35 to 41, wherein the patient has received a combination of the vaccines.

43. The method of any one of claims 1 to 42, wherein the patient has experienced one or more prior infections by or exposures to the virus.

44. The method of claim 43, wherein the patient has experienced one prior infection by or exposure to the virus.

45. The method of claim 43, wherein the patient has experienced two or more prior infections by or exposures to the virus.

46. The method of claim 43, wherein the patient has experienced three or more prior infections by or exposures to the virus.

47. The method of claim 43, wherein the patient has experienced one or more prior infections by or exposures to the virus of at least about two weeks before the breakthrough infection.

48. The method of any one of claims 1 to 47, wherein the patient is afflicted with one or more of fever, cough, shortness of breath, diarrhea, upper respiratory symptoms, lower respiratory symptoms, pneumonia, and acute respiratory syndrome.

49. The method of any one of claims 1 to 48, wherein the patient is afflicted with fever, tiredness, dry cough, aches and pains, shortness of breath and other breathing difficulties, diarrhea, upper respiratory symptoms (e.g., sneezing, runny nose, nasal congestion, cough, sore throat), pneumonia, pneumonia respiratory failure, hepatic and renal insufficiency, acute respiratory distress syndrome (ARDS), and a cytokine imbalance by one or more symptoms of a coronavirus infection relative to an uninfected state.

50. The method of claim 48 or claim 49, wherein the patient has mild or moderate COVID-19.

51. The method of any one of the claims 48 to 50, wherein the patient is elderly and/or afflicted with one or more comorbidities.

52. The method of any one of the claims 48 to 50, wherein the patient has one or more characteristics which provide a high risk for progression to severe disease.

53. The method of any one of the claims 48 to 50, wherein the patient has one or more pre-existing medical conditions that reduce an immune response.

54. The method of any one of the claims 48 to 50, wherein the patient is overweight or obese.

55. The method of any one of claims 1 to 54, where the method corrects an immune imbalance in the subject.

56. The method of any one of claims 1 to 55, wherein the method causes an increase in the humoral response.

57. The method of any one of claims 1 to 56, wherein the method causes an increase in an antibody response.

58. The method of any one of claims 1 to 57, wherein the method causes a change in the kinetics and/or magnitude of an antibody response.

59. The method of any one of claims 1 to 57, wherein the method causes an increase in one or more antibody isotypes, optionally selected from immunoglobulin M (IgM), immunoglobulin G (IgG), immunoglobulin A (lgA), immunoglobulin D (IgD) and immunoglobulin E (lgE).

60. The method in claim 59, wherein the antibody isotype is IgM.

61. The method in claim 59, wherein the antibody isotype is IgG.

62. The method in claim 61, wherein the IgG is of subclass IgG4.

63. The method of any one of claims 1 to 62, wherein the method induces antibody isotype class switching.

64. The method in claim 63, wherein the immunoglobulin isotype class switch is from IgM isotype to IgG isotype, optionally selected from IgG subclasses IgG1, IgG2, IgG3 and IgG4.

65. The method in claim 64, wherein the isotype class switch is from IgM to IgG4.

66. The method of any one of claims 1 to 65, wherein the method prevents or mitigates the development or progression of the infection in the patient.

67. The method of any one of claims 1 to 66, wherein the method prevents or mitigates the development or progression of one or more of fever, cough, shortness of breath, diarrhea, upper respiratory symptoms, lower respiratory symptoms, pneumonia, and acute respiratory syndrome.

68. The method of any one of claims 1 to 67, wherein the method prevents or mitigates the development or progression of one or more of fever, tiredness, dry cough, aches and pains, shortness of breath and other breathing difficulties, diarrhea, upper respiratory symptoms (e.g., sneezing, runny nose, nasal congestion, cough, sore throat), pneumonia, pneumonia respiratory failure, hepatic and renal insufficiency, acute respiratory distress syndrome (ARDS), a cytokine and an immune imbalance.

69. The method of any one of claims 1 to 68, wherein the method partially inhibits an immune response to reduce or ablate a cytokine imbalance in the subject.

70. The method of any one of claims 1 to 69, wherein the method reverses, reduces or prevents a cytokine storm.

71. The method of any one of claims 1 to 70, wherein the method reverses, reduces or prevents a cytokine storm in the lungs.

72. The method of any one of claims 1 to 71, wherein the method prevents or mitigates the development or progression of the infection that requires hospitalization.

73. The method of any one of claims 1 to 72, wherein the method prevents or mitigates the development or progression of the infection that causes death of the patient.

74. The method of any one of claims 1 to 73, wherein the method prevents or mitigates the development or progression of the infection that requires receiving invasive mechanical ventilation.

75. The method of any one of claims 1 to 74, wherein the method prevents or mitigates the development or progression of the infection that requires receiving extracorporeal membrane oxygenation (ECMO).

76. The method of any one of claims 1 to 75, wherein the method prevents or mitigates the development or progression of the infection that requires receiving non-invasive mechanical ventilation.

77. The method of any one of claims 1 to 76, wherein the method prevents or mitigates the development or progression of the infection that requires use of a high flow oxygen device.

78. The method of any one of claims 1 to 77, wherein the method prevents or mitigates the development or progression of the infection that requires receiving supplemental oxygen.

79. The method of any one of claims 1 to 78, wherein the method prevents or mitigates the development or progression of the infection that requires receiving ongoing medical care.

80. The method of any one of claims 1 to 79, wherein the method prevents or mitigates the development or progression of the infection to a NIAID Ordinal Scale score of 3 to 8.

81. The method of any one of claims 1 to 80, wherein the method prevents or mitigates the development or progression of the infection to an NIAID Ordinal Scale score of 8.

82. The method of any one of claims 1 to 80, wherein the method prevents or mitigates the development or progression of the infection to an NIAID Ordinal Scale score of 7.

83. The method of any one of claims 1 to 80, wherein the method prevents or mitigates the development or progression of the infection to an NIAID Ordinal Scale score of 6.

84. The method of any one of claims 1 to 80, wherein the method prevents or mitigates the development or progression of the infection to an NIAID Ordinal Scale score of 5.

85. The method of any one of claims 1 to 80, wherein the method prevents or mitigates the development or progression of the infection to a NIAID Ordinal Scale score of 4.

86. The method of any one of claims 1 to 80, wherein the method prevents or mitigates the development or progression of the infection to a NIAID Ordinal Scale score of 3.

87. The method of any one of claims 1 to 86, wherein the method prevents or mitigates the development or progression of the infection as measured by patient global assessment scores (e.g., Clinical Global Impression (CGI) scale).

88. The method of any one of claims 1 to 87, wherein the method improves symptoms and functional abilities in a patient as measured by patient global assessment scores (e.g., Clinical Global Impression (CGI) scale).

89. The method of any one of claims 1 to 88, wherein the method prevents or mitigates the development or progression of the infection that requires receiving an additional therapeutic agent.

90. The method of claim 89, wherein the additional therapeutic agent is selected from nirmatrelvir/ritonavir, remdesivir; favipiravir; galidesivir; prezcobix; lopinavir; and/or ritonavir; and/or arbidol lopinavir/ritonavir; and/or ribavirin; and/or IFN-beta; xiyanping; anti-VEGF-A; fingolimod; carrimycin; hydroxychloroquine; darunavir and cobicistat; methylprednisolone; brilacidin; leronlimab; thalidomide, bamlanivimab, casirivimab, and imdevimab.

91. The method of claim 90, wherein the additional therapeutic agent is nirmatrelvir/ritonavir (PAXLOVID).

92. The method of claim 89, wherein the additional therapeutic agent is an antibody directed against an antigen of the virus.

93. The method of claim 92, wherein the additional therapeutic agent is a monoclonal antibody directed against a coronavirus antigen.

94. The method of claim 89, wherein the additional therapeutic agent is a steroid.

95. The method of claim 94, wherein the steroid is dexamethasone or prednisone.

96. The method of any one of claims 1 to 95, wherein the method causes a decrease in viral load in the patient relative to before treatment.

97. The method of any one of claims 1 to 96, wherein the method restores the cytokine balance and restores homeostasis in the patient relative to before treatment.

98. The method of claim 97, wherein the cytokine is one or more of is one or more of IL-6, IL-1, IL-1 receptor antagonist (IL-1ra), IL-2ra, IL-10, IL-18, TNFα, interferon-g (IFN-g), CXCL10, and CCL7.

99. The method of claim 98, wherein the cytokine is IL-10.

100. The method of claim 98, wherein the cytokine is interferon-gamma (IFN-g).

101. The method of any one of claims 1 to 100, wherein the method reduces or mitigates a likelihood of the patient developing ARDS.

102. The method of any one of claims 1 to 101, wherein the method reduces or mitigates a likelihood of the patient developing lung fibrosis.

103. The method of any one of claims 59 to 102, wherein the method reduces or mitigates a likelihood of the patient developing long COVID.

104. The method of any one of claims 1 to 103, wherein the breakthrough viral infection is assayed or assayable in a biological sample from the patient.

105. The method of claim 104, wherein the biological sample is or comprises respiratory fluid, sputum, saliva, or stool.

106. The method of claim 105, wherein the respiratory fluid is from an oropharyngeal (OP) or nasopharyngeal (NP) swab.

107. The method of claim 105, wherein the respiratory fluid is or comprises a nasal secretion.

108. The method of claim 105, wherein the respiratory fluid is or comprises saliva.

109. The method of claim 104, wherein the breakthrough viral infection is assayed or assayable using a measurement of a viral nucleic acid or protein.

110. The method of claim 104, wherein the breakthrough viral infection is assayed or assayable using PCR.

111. The method of claim 104, wherein the breakthrough viral infection is assayed or assayable using a rapid test.

112. The method of any one of claims 1 to 111, wherein the GM-CSF has an amino acid sequence of SEQ ID NO: 1, or a variant of at least about 90%, or at least about 93%, or at least about 95%, or at least about 97%, or at least about 98% identity thereto.

113. The method of any one of claims 1 to 111, wherein the GM-CSF has an amino acid sequence of SEQ ID NO: 2 or SEQ ID NO: 3, or a variant of at least about 90%, or at least about 93%, or at least about 95%, or at least about 97%, or at least about 98% identity thereto.

114. The method of any one of claims 1 to 113, wherein the GM-CSF is one of molgramostim, sargramostim, and regramostim.

115. The method of claim 114, wherein the GM-CSF is sargramostim.

116. The method of any one of claims 1 to 113, wherein the GM-CSF is administered at a total dose of about 125 μg, about 150 μg, or about 200 μg, or about 250 μg, or about 300 μg, or about 350 μg.

117. The method of claim 116, wherein the GM-CSF is administered at a total dose of about 250 μg.

118. The method of any one of claims 1 to 115, wherein the GM-CSF is administered at a dose of about 125 μg, about 150 μg, or about 200 μg, or about 250 μg, or about 300 μg, or about 350 μg, once daily.

119. The method of claim 118, wherein the GM-CSF is sargramostim, administered at a dose of about 250 μg, once daily.

120. The method of any one of claims 1 to 115, wherein the GM-CSF is sargramostim, administered at a dose of about 250 μg, once daily, for 5 consecutive days.

121. The method of claim 120, wherein the GM-CSF is sargramostim, administered at a dose of about 250 μg, once daily, for 5 consecutive days following a positive test for the viral infection or the start of symptoms of the viral infection.

122. The method of claim 120, wherein the GM-CSF is sargramostim, administered at a dose of about 250 μg, once daily, for 5 consecutive days following a positive test for COVID-19.

123. The method of any one of claims 1 to 122, wherein the GM-CSF is administered by inhalation.

124. The method of any one of claims 1 to 122, wherein the GM-CSF is administered to the lung.

125. The method of claim 124, wherein the GM-CSF is administered via aerosol or nebulizer.

126. The method of claim 125, wherein the aerosol or nebulizer is selected from liquid nebulization, dry powder dispersion and meter-dose administration.

127. The method of any one of claims 1 to 126, wherein the method further comprises administering one or more additional therapeutic agents.

128. The method of claim 127, wherein the additional therapeutic agent is selected from nirmatrelvir/ritonavir, remdesivir; favipiravir; galidesivir; prezcobix; lopinavir; and/or ritonavir; and/or arbidol lopinavir/ritonavir; and/or ribavirin; and/or IFN-beta; xiyanping; anti-VEGF-A; fingolimod; carrimycin; hydroxychloroquine; darunavir and cobicistat; methylprednisolone; brilacidin; leronlimab; thalidomide, bamlanivimab, casirivimab, and imdevimab.

129. The method of claim 128, wherein the additional therapeutic agent is nirmatrelvir/ritonavir (PAXLOVID).

130. The method of claim 127, wherein the additional therapeutic agent is an antibody directed against an antigen of the virus.

131. The method of claim 130, wherein the additional therapeutic agent is a monoclonal antibody directed against a coronavirus antigen.

132. The method of claim 127, wherein the additional therapeutic agent is a steroid.

133. The method of claim 132, wherein the steroid is dexamethasone or prednisone.

134. The method of any one of claims 1 to 133, wherein the method further comprises the step of monitoring the viral load during the course of treatment.

135. The method of claim 134, wherein the monitoring of viral load is assayed or assayable using a measurement of viral nucleic acid or protein.

136. The method of claim 134, wherein the monitoring of viral load is assayed or assayable using PCR.

137. The method of claim 134, wherein the monitoring of viral load is assayed or assayable using a rapid test.

138. The method of claim 134, wherein the monitoring of viral load is assayed or assayable using a lateral flow antigen test.

139. The method of any one of claims 1 to 138, wherein an increase in viral load in the patient directs continued administration of GM-CSF.

140. The method of any one of claims 1 to 138, wherein a worsening of symptoms in the patient with a viral infection at any timepoint directs toward continued administration of the GM-CSF.

141. The method of any one of claims 1 to 138, wherein a worsening in the NIAID Ordinal Scale score in a patient with a viral infection at any timepoint directs toward continued administration of GM-CSF.

142. The method of any one of claims 1 to 138, wherein a decrease in viral load directs towards discontinuation of administration of GM-CSF or a reduction in dosage of GM-CSF.

143. The method of any one of claims 1 to 138, wherein improvement in patient global assessment scores (e.g., Clinical Global Impression (CGI) scale) in a patient with a viral infection at any timepoint directs discontinuation of administration of GM-CSF a reduction in dosage of GM-CSF.
